(19) United States
(12) Patent Application Publication
(10) Pub. No.: US 2026/0318896 A1
(43) Pub. Date: Oct. 15, 2026

(54) CENTRIFUGE TEST DEVICE AND METHOD FOR COMPLEX FAULT ACTIVATION IN WATER-RICH TUNNELS INDUCED BY EARTHQUAKES/CREEP

(71) Applicants: GUANGXI UNIVERSITY, Nanning (CN); Tongji University, Shanghai (CN); Institute of Rock and Soil Mechanics, Chinese Academy of Sciences, Wuhan (CN); State Key Laboratory of Shield Machine and Boring Technology, Zhengzhou (CN)

(72) Inventors: Zhi ZHENG, Nanning (CN); Wuqiang CAI, Nanning (CN); Pengzhi PAN, Nanning (CN); Quan JIANG, Nanning (CN); Bingrui CHEN, Nanning (CN); Tiejun TAO, Nanning (CN); Zhaofeng WANG, Nanning (CN); Dingping XU, Nanning (CN); Shili QIU, Nanning (CN); Hong ZHENG, Nanning (CN); Gaoming LU, Nanning (CN); Zhanpeng MA, Nanning (CN)

(21) Appl. No.: 19/278,564

(22) Filed: Jul. 23, 2025

(30) Foreign Application Priority Data
Apr. 11, 2025 (CN) 202510450867.5

(51) Int. Cl. G01N 33/24

(52) U.S. Cl. CPC G01N 33/24; G01N 2203/0037; G01N 2203/0064; G01N 2203/0071; G01N 2203/0256

(57) ABSTRACT

A centrifuge test device for complex fault activation in water-rich tunnels induced by earthquakes/creeps includes an overload loading system, an earthquake wave loading system and a true triaxial loading device; the overload loading system includes a centrifuge main body, a test basket and a counterweight block; the earthquake wave loading system is composed of two identical vibration tables, the vibration table includes a vibration table base, a vibration exciter, a vibration table linear guide rail, a vibration table linear guide slider and a vibration table surface; the true triaxial loading device includes an external frame, an internal sealing frame and an earthquake/creep simulation test body.

### CROSS-REFERENCE TO RELATED APPLICATIONS

This application claims priority to Chinese Patent Application No. 202510450867.5, filed on Apr. 11, 2025, the entire disclosure of which is incorporated herein by reference.

### TECHNICAL FIELD

The present application relates to the technical field of geotechnical engineering and test device, in particular to a centrifuge test device and method for complex fault activation in water-rich tunnels induced by earthquakes/creep.

### BACKGROUND

In recent years, China's infrastructure construction focus has gradually shifted to the western and southwest regions. The geological conditions in these regions are complex and varied. During the construction of tunnels, they will pass through some geologically active zones, and some will even go through faults with earthquake activity. A large number of major national projects such as the Pinglu Canal are distributed in mountainous regions with active tectonics and well-developed faults. The routes often cross active faults and have large burial depths at the same time. Fault creep refers to the slow, non-earthquake sliding of natural tectonic faults. Therefore, studying the activation of complex faults in water-rich tunnels induced by earthquakes/creep has become an important scientific issue. During an earthquake, the stress state in the crust changes sharply, leading to fault sliding and creep, which in turn affects the stability and safety of the tunnel. Especially in water-rich regions, the action of groundwater will further intensify the interaction between the tunnel and the fault, making the problem more complicated.

At present, research on the activation of complex faults in water-rich tunnels induced by earthquakes/creep mainly relies on numerical simulations and theoretical analyses. However, these methods often cannot fully reflect the complexity and nonlinear characteristics in actual engineering, so the test results have certain limitations and uncertainties. Traditional test device and methods have many limitations in simulating the earthquake environment and tunnel structure. Traditional centrifuge device can only simulate the activation of faults with a specific occurrence, and there is a lack of device that can simulate the three-dimensional in-situ stress, overweight, groundwater occurrence environment, and the coupling conditions of different fault occurrences to induce fault slip. There is a lack of device that can simulate the activation of different fault types such as normal/reverse faults and horizontal faults, and faults with different occurrences such as different fault dips under the action of three-dimensional in-situ stress, earthquake force, and groundwater occurrence environment. The traditional tunnel surface force loading test system cannot simulate the differences in tunnel disasters caused by different principal stress directions at different depths, and the existing device cannot simulate the response of tunnels under the multi-field coupling environment of true three-dimensional stress, water-rich conditions, earthquake/creep, and overweight. Therefore, developing a test device and method that can simulate the activation of complex faults in water-rich tunnels under real earthquake/creep environments is of great significance for improving the stability and safety of tunnel projects.

### SUMMARY

In view of the problems existing in the related art, the present application provides a centrifuge test device and method for complex fault activation in water-rich tunnels induced by earthquakes/creep, which can be used to carry out indoor centrifuge tests on complex fault activation in water-rich tunnels induced by earthquakes/creep, so as to realize the test simulation of tunnel samples under the multi-field coupling environment of true three-dimensional stress, water-rich conditions, earthquakes, overweight and the like.

To achieve the above purpose, the present application adopts the following technical scheme:

A centrifuge test device for complex fault activation in water-rich tunnels induced by earthquakes/creep, comprising an overweight loading system, an earthquake wave loading system, a true triaxial loading device for complex fault tunnels, a physical model of a composite-lined fault tunnel, and a comprehensive monitoring system for deformation-stress-vibration;
the overweight loading system comprises a centrifuge main body, a test basket, and a counterweight block;
the earthquake wave loading system comprises a lower vibration table base, a lower exciter, a lower vibration table linear guide rail, a lower vibration table linear guide slider, an upper vibration table base, an upper exciter, an upper vibration table linear guide rail, an upper vibration table linear guide slider, a vibration table surface, and a stop block;
the true triaxial loading device for complex fault tunnels comprises a main frame, a top cover, an oil cylinder, an inner pressure-bearing sealing frame, a pressure head, and a backing plate;
the physical model of the composite-lined fault tunnel comprises a tunnel sample, a lining, a sheath, and an inner lining plug;
the comprehensive monitoring system for deformation-stress-vibration comprises a pre-embedded optical fiber, a water pressure sensor, a pressure sensor, and a vibration wave monitor;
the centrifuge test device for complex fault activation in water-rich tunnels induced by earthquakes/creep activates a fault of the tunnel sample by adjusting a pressure on the upper and lower sides of the tunnel sample, further causing a tunnel sample to creep along the fault;
the centrifuge main body provides overweight centrifugal force through high-speed rotation; the test basket is provided on both sides of the centrifuge main body;
the counterweight block and the vibration table are respectively provided on the two test baskets; the lower vibration table base is fixed on the test basket of the centrifuge;
the lower exciter is provided and fixed on the lower vibration table base, with both ends attached to the upper vibration table base; the lower vibration table linear guide rail is provided and fixed on the lower vibration table base; one end of the lower vibration table linear guide slider is provided on the lower vibration table linear guide rail, and another end of the lower vibration table linear guide slider is provided and fixed on a lower part of the upper vibration table base;
the upper vibration table base is connected to a table surface of the lower vibration table through the lower vibration table linear guide rail; the upper exciter is provided and fixed on the upper vibration table base, with both ends attached to a table surface of the upper vibration table;
the upper vibration table linear guide rail is provided and fixed on the upper vibration table base; one end of the upper vibration table linear guide slider is provided on the upper vibration table linear guide rail, and another end of the upper vibration table linear guide slider is provided and fixed on the lower part of the table surface of the upper vibration table;
the table surface of the upper vibration table is connected to the upper vibration table base through the upper vibration table linear guide slider; the table surface of the upper vibration table is connected to the true triaxial loading device for complex fault tunnels.

An experimental method of the centrifuge test device for complex fault activation in water-rich tunnels induced by earthquakes/creep as mentioned above, includes
Step 1: making the tunnel sample;
Step 2: installing the lining, the sheath, the sensor, and the monitoring camera into the tunnel sample, injecting an appropriate amount of water into a space formed by the sheath, then installing inner lining plugs on both sides of the sheath to form the physical model of the composite-lined fault tunnel, ensuring that the inner lining plugs completely seal the tunnel;
Step 3: using lifting device to load the physical model of the composite-lined fault tunnel into the inner sealing frame, and selecting a suitable backing plate, so that a fault dip angle of the physical model of the composite-lined fault tunnel after being loaded into the inner sealing frame meets test requirements;
Step 4: injecting water into the inner pressure-bearing sealing frame, covering the top plate of the inner pressure-bearing sealing frame tightly, making the pressure head in close contact with the physical model of the composite-lined fault tunnel, and checking a tightness of the inner pressure-bearing frame;
Step 5: covering the top cover of the outer frame tightly, and ensuring that the upper oil cylinder is connected to an upper pressure head of the inner sealing frame;
Step 6: if simulating an activation phenomenon of normal and reverse faults in water-rich tunnels induced by earthquakes and three-dimensional in-situ stress, the following steps are adopted:
   a. starting the oil cylinder on the true triaxial loading device to apply pre-pressure to the physical model of the composite-lined fault tunnel;
   b. reading the data of the wireless sensor and the monitoring camera to determine whether the wireless sensor and the monitoring camera are working normally;
   c. applying confining pressure, applying the same confining pressure on upper and lower sides through the oil cylinder on the upper and lower sides, and applying confining pressure that increases from top to bottom due to different depths in other directions;
   d. after applying the confining pressure, observing a creep of the physical model of the composite-lined fault tunnel, starting the exciter of the vibration table after a period of time, making a table surface of the vibration table vibrate at high frequency through the exciter, and the table surface of the vibration table applying earthquake waves to the physical model of the composite-lined fault tunnel to simulate the activation of normal and reverse faults in water-rich tunnels induced by earthquakes and three-dimensional in-situ stress;
Step 7: if simulating an activation phenomenon of horizontal faults in water-rich tunnels induced by overweight and three-dimensional stress, the following steps are adopted:
   a. starting the oil cylinder on the true triaxial loading device to apply pre-pressure to the physical model of the composite-lined fault tunnel to prevent an overall sliding of the physical model of the composite-lined fault tunnel during a centrifuge test;
   b. applying confining pressure according to a test plan;
   c. after applying the confining pressure, reading the data of the wireless sensor and the monitoring camera to determine whether the wireless sensor and the monitoring camera are working normally;
   d. starting the centrifuge, applying a rotation speed within 10 g, and observing whether there are potential safety hazards in the test;
   e. slowly increasing a rotation speed of the centrifuge to a target speed to simulate the activation of horizontal faults in water-rich tunnels induced by overweight and three-dimensional stress;
Step 8: after the physical model of the composite-lined fault tunnel reaches an expected test condition or is damaged, the experiment is ended, and stopping the earthquake table and the centrifuge, to reduce the pressure to zero, turning off the centrifuge and the vibration table, turning off the true triaxial loading device, and overhauling;
Step 9: analyzing and processing according to detection data recorded by the wireless sensor and the monitoring camera.

The beneficial effects of the present application are as follows:

The present application can simulate a water-rich fault-containing tunnel sample under a three-dimensional stress state, and can activate the fault in the tunnel sample by adjusting the pressure on the upper and lower sides of the tunnel sample, further causing the tunnel sample to creep along the fault, thereby studying the impact of tunnel fault creep on the tunnel and its lining. The water-rich fault-containing tunnel sample of the present application can undergo fault activation triggered by earthquake forces generated by the vibration table under the action of three-dimensional stress, thus simulating the extrusion damage to the lining caused by the slip of the tunnel sample and the impact of water-related disasters. The present application can simulate the creep failure of faults in the tunnel sample under the overweight effect generated by the high-speed rotation of the centrifuge. The present application can realize activation simulation tests for faults with different occurrences, as well as creep simulation tests for normal/reverse faults and horizontal faults. The present application can simulate the impact of water disasters on the lining structure in the tunnel caused by fault creep under water-covered conditions, as well as the responses of the lining and surrounding rock under multi-field coupling activation in water-rich environments. Through multi-field coupling of true three-dimensional stress, water-rich conditions, earthquakes, and overweight, the present application achieves a high degree of restoration of the real environment of the tunnel sample in the laboratory, which is of great significance for improving the stability and safety of tunnel engineering.

Other features and advantages of the present application will be described in detail in the specific embodiments below.

### BRIEF DESCRIPTION OF THE DRAWINGS

FIG. 1 is an overall structural diagram of the centrifuge test device for complex fault activation in water-rich tunnels induced by earthquakes/creep according to the present application.

FIG. 2 is a combined structural diagram of an earthquake wave loading system and a true triaxial loading device for complex fault tunnels of the centrifuge test device for complex fault activation in water-rich tunnels induced by earthquakes/creep according to the present application.

FIG. 3 is a half-sectional view of a combined structure of the earthquake wave loading system and the true triaxial loading device for complex fault tunnels of the centrifuge test device for complex fault activation in water-rich tunnels induced by earthquakes/creep according to the present application.

FIG. 4 is a front sectional view of the combined structure of the earthquake wave loading system and the true triaxial loading device for complex fault tunnels of the centrifuge test device for complex fault activation in water-rich tunnels induced by earthquakes/creep according to the present application.

FIG. 5 is a side sectional view of the combined structure of the earthquake wave loading system and the true triaxial loading device for complex fault tunnels of the centrifuge test device for complex fault activation in water-rich tunnels induced by earthquakes/creep according to the present application.

FIG. 6 is a horizontal sectional view of the combined structure of the earthquake wave loading system and the true triaxial loading device for complex fault tunnels of the centrifuge test device for complex fault activation in water-rich tunnels induced by earthquakes/creep according to the present application.

FIG. 7 is a structural schematic diagram of the earthquake wave loading system of the centrifuge test device for complex fault activation in water-rich tunnels induced by earthquakes/creep according to the present application.

FIG. 8 is a sectional schematic diagram of the true triaxial loading device for complex fault tunnels of the centrifuge test device for complex fault activation in water-rich tunnels induced by earthquakes/creep according to the present application.

FIG. 9 is a structural schematic diagram of an inner sealing frame of the centrifuge test device for complex fault activation in water-rich tunnels induced by earthquakes/creep according to the present application.

FIG. 10 is a structural diagram of a physical model of the composite-lined fault tunnel of the centrifuge test device for complex fault activation in water-rich tunnels induced by earthquakes/creep according to the present application.

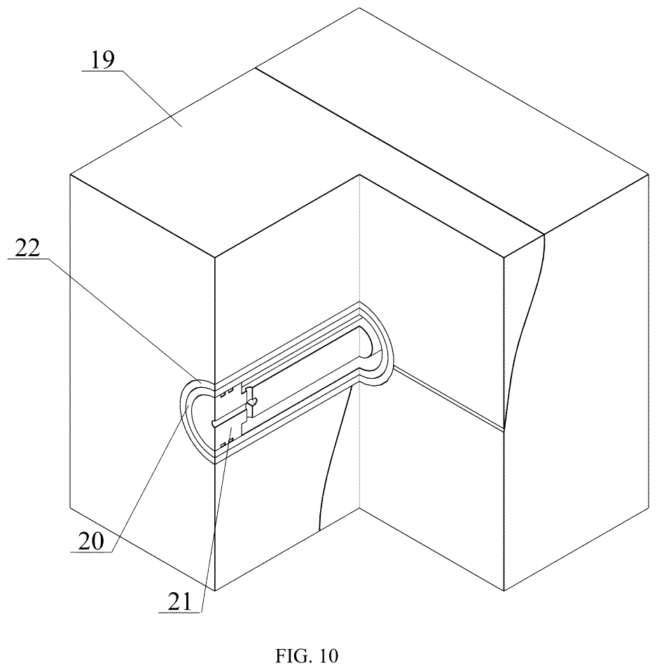

FIG. 11 is a stress distribution diagram of the first embodiment of the present application.

FIG. 12 is a stress distribution diagram of the second embodiment of the present application.

### DETAILED DESCRIPTION OF THE EMBODIMENTS

The present application will be further described in detail below with reference to the accompanying drawings and embodiments. It is obvious that the described embodiments are only a part of the embodiments of the present application, not all of them.

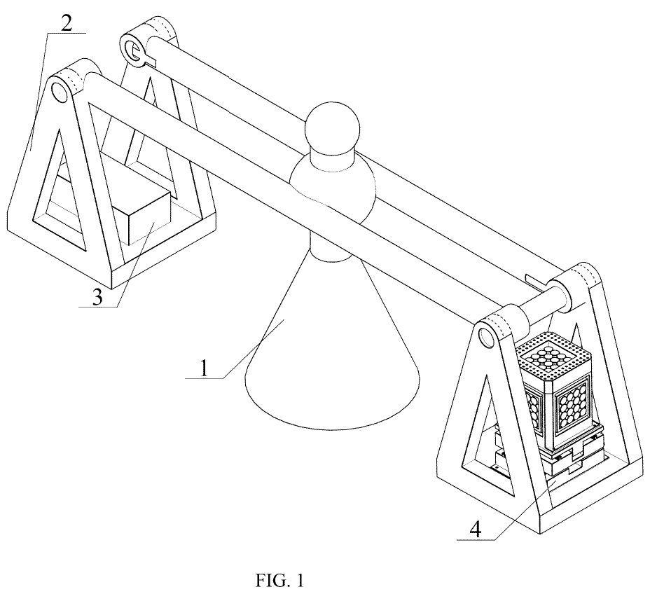

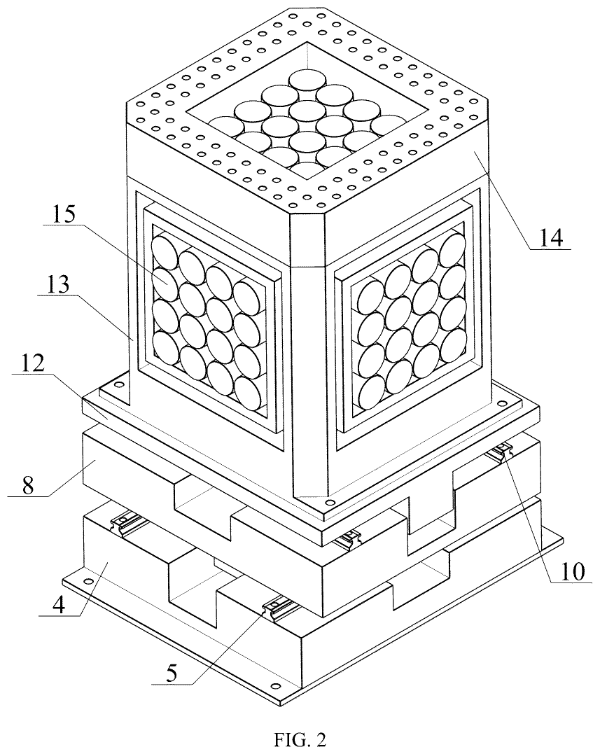

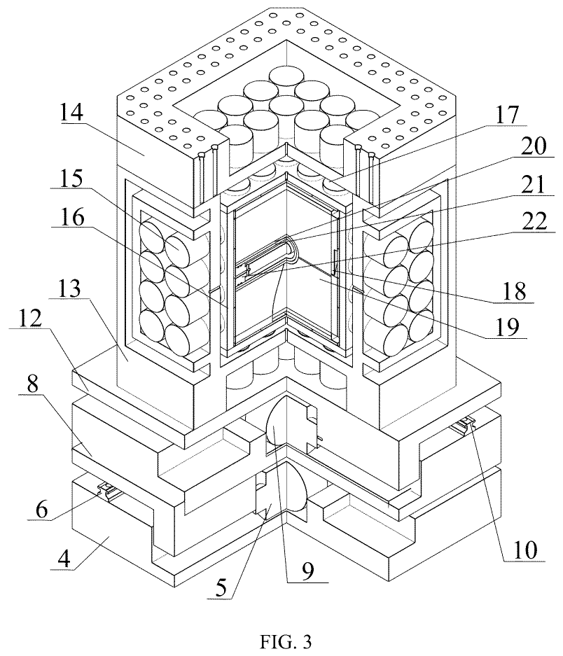

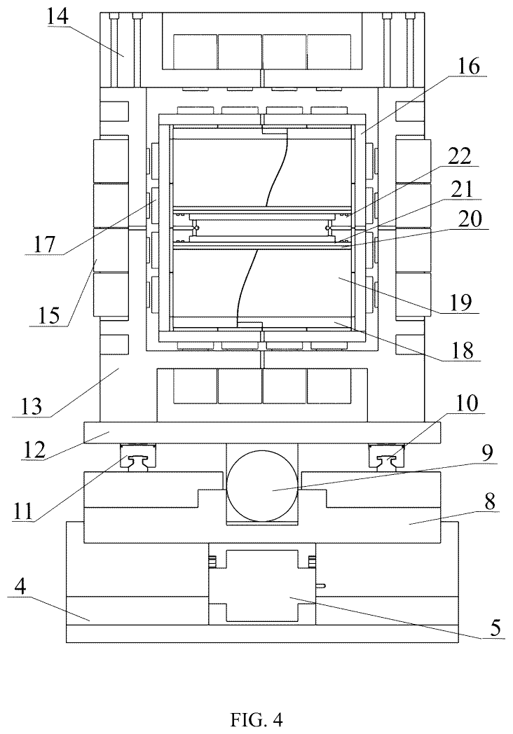

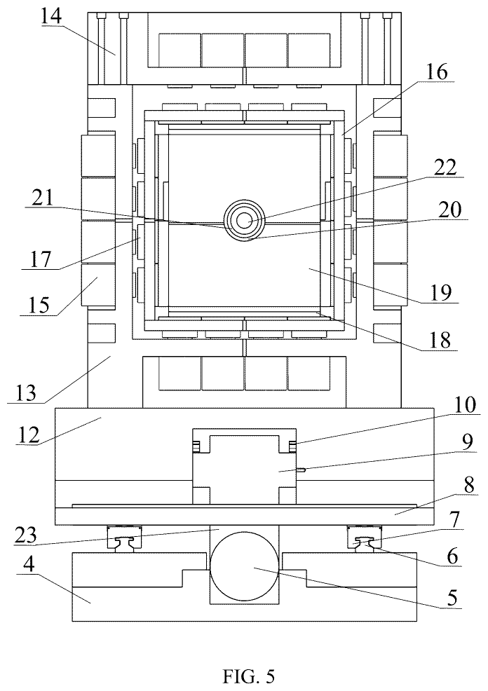

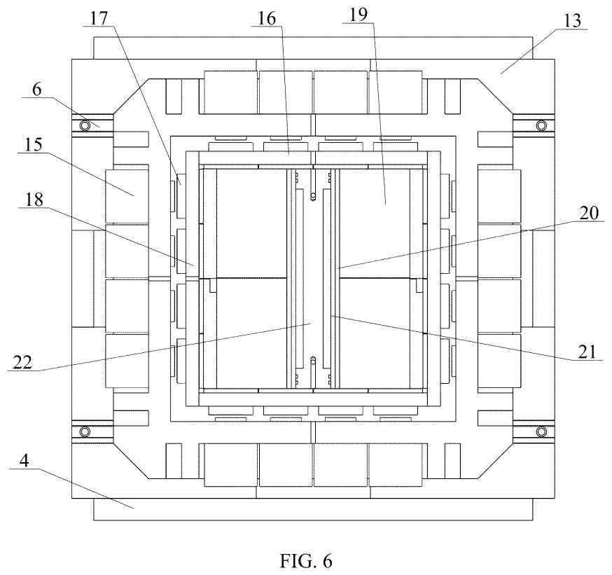

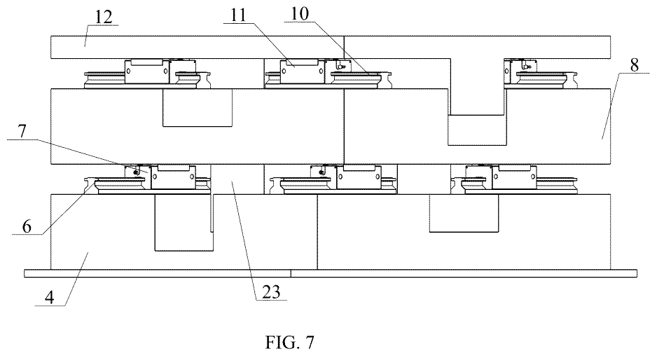

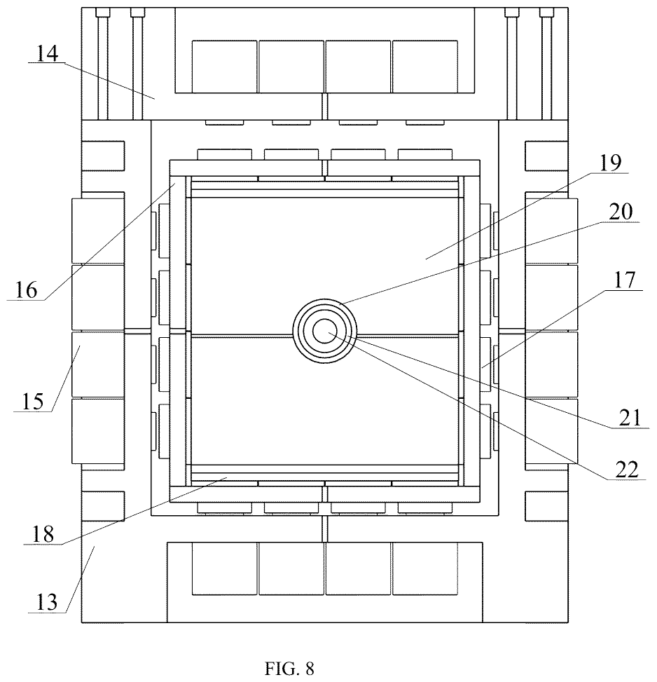

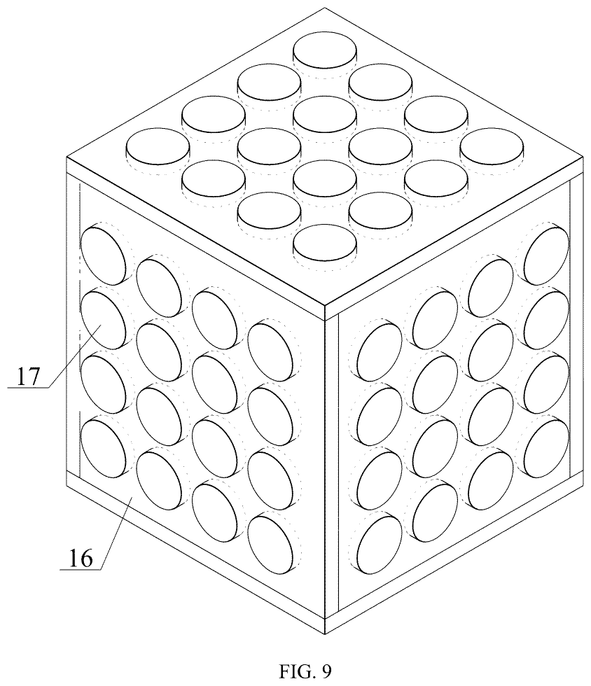

To solve the problems existing in the related art, as shown in FIGS. 1-9, the present application provides a centrifuge test device for complex fault activation in water-rich tunnels induced by earthquakes/creep, which includes an overweight loading system, an earthquake wave loading system, a true triaxial loading device for complex fault tunnels, a physical model of composite-lined fault tunnels, and a comprehensive monitoring system for deformation-stress-vibration.

The overweight loading system includes a centrifuge main body 1, a test basket 2 and a counterweight block 3; a bottom of the centrifuge main body 1 is deeply buried underground, and provides overweight centrifugal force through high-speed rotation for realizing overweight loading during the test. The test baskets 2 are provided on both sides of the centrifuge main body 1, which can be in horizontal and vertical states, and are used for installing the overweight loading system and the physical model of composite-lined fault tunnels. the counterweight block 3 and the vibration table are respectively provided on two test basket 2 for counterweight during the test.

The earthquake wave loading system includes a lower vibration table base 4, a lower exciter 5, a lower vibration table linear guide rail 6, a lower vibration table linear guide slider 7, an upper vibration table base 8, an upper exciter 9, an upper vibration table linear guide rail 10, an upper vibration table linear guide slider 11, a vibration table surface 12 and a stop block 23. the lower vibration table base 4 is provided and fixed on one test basket 3 of the centrifuge, and is used for installing and fixing other structures of the vibration table and the true triaxial loading device for complex fault tunnels; the lower exciter 5 is provided on the lower vibration table base 4, and both ends are limited by the stop blocks 23, and the lower exciter 5 is connected with a control terminal for generating and sending earthquake waves in one direction; the lower vibration table linear guide rail 6 is provided and fixed on the lower vibration table base 4. One end of the lower vibration table linear guide slider 7 is provided on the lower vibration table linear guide rail 6, and another end of the lower vibration table linear guide slider 7 is provided and fixed on the lower part of the upper vibration table base 8. The lower vibration table linear guide rail 6 and the lower vibration table linear guide slider 7 form a lower vibration table linear guide, which is used to connect the lower vibration table base 4 and the upper vibration table base 8, and used to transmit earthquake waves to the upper vibration table base 8 when the lower exciter 5 applies earthquake waves. The upper vibration table base 8 is connected with the lower vibration table base 4 through the lower vibration table linear guide rail, and is used to transmit earthquake waves in one direction. The upper exciter 9 is provided on the upper vibration table base 8, and the upper exciter 9 is connected with the control terminal for generating and sending earthquake waves in one direction. The upper vibration table linear guide rail 10 is provided and fixed on the upper vibration table base 8. One end of the upper vibration table linear guide slider 11 is provided on the lower vibration table linear guide rail 10, and another end of the upper vibration table linear guide slider 11 is provided and fixed on the lower part of the upper vibration table surface 12. The vibration table surface 12 of the upper vibration table is connected with the main frame 13 of the true triaxial loading device for complex fault tunnels.

The true triaxial loading device for complex fault tunnels includes a main frame 13, a top cover 14, an oil cylinder 15, an inner pressure-bearing sealing frame 16, a pressure head 17 and a backing plate 18. The main frame 13 is provided and fixed on a vibration table surface 8 of the upper vibration table, and is used to connect the overweight loading system and the true triaxial loading device for complex fault tunnels. The top cover 14 is provided on the upper side of the main frame 13, and forms an installation space for the inner sealing frame together with the main frame 13. The oil cylinder 15 are provided and fixed in a reserved hole of the main frame 13 and the top cover 14. 16 oil cylinders 15 are provided and fixed on each side of the frame structure formed by the main frame 13 and the top cover, and the oil cylinders 15 are connected with the control terminal for applying loads in various directions during the test. When the oil cylinders 15 of the outer frame apply loads, different oil cylinders 15 in the same direction can apply different forces or the same force. The inner pressure-bearing sealing frame 16 is provided in the closed space formed by the main frame 13, and is used to install the physical model of the composite-lined fault tunnel and realize sealing under water-rich conditions. The pressure head 17 is provided in the reserved hole of the inner pressure-bearing seal 11 and connected with the oil cylinder 15, and is used to apply the load generated by the oil cylinder 15 to the physical model of the composite-lined fault tunnel. The backing plate 18 is provided in the inner pressure-bearing sealing frame 16 and is attached to the tunnel sample 19, the inclination angle of the tunnel sample 19 can be changed by replacing the backing plate 18, and the stress region of the tunnel sample 19 can also be changed by adjusting the placement position of the backing plate 18.

The physical model of the composite-lined fault tunnel includes a tunnel sample 19, a lining 20, a sheath 21 and an inner lining plug 22; the tunnel sample 19 is a scaled-down model of an actual tunnel containing faults provided in the inner pressure-bearing sealing frame, and the scaling ratio of the model should be greater than a maximum overweight multiple generated by the centrifuge. The lining 20 is provided in the tunnel sample close to the tunnel sample for maintaining the stability of the tunnel sample 19. The sheath 21 is provided in the tunnel sample 19 close to the lining 20 for isolating the connection between the lining 20 and the external environment, reducing the corrosion degree of the lining 20 under water-rich conditions, and maintaining the stability of the tunnel sample 19 to a certain extent. The inner lining plug 22 is provided in the space formed by the sheath 21 to form a closed space with the sheath 21 to isolate internal water from external water, and is used to seal the tunnel opening of the tunnel sample 19 to prevent the water injected into the inner sealing frame during simulating water-rich conditions from entering the tunnel, which would lead to a large deviation between the test results and the actual project. Wireless sensors are provided between the tunnel sample 19 and the lining 20, and between the lining 20 and the sheath 21 for monitoring the real-time status of the physical model of the composite-lined fault tunnel. A monitoring camera is provided in the closed space formed by the sheath 21 and the inner lining plug 22 for recording the internal status of the physical model of the composite-lined fault tunnel during the test.

The comprehensive monitoring system for deformation-stress-vibration monitors the deformation, stress and response under vibration waves of the physical model of the composite-lined fault tunnel. The comprehensive monitoring system for deformation, stress and vibration includes a pre-embedded optical fiber 24, a water pressure sensor 25, a pressure sensor 26 and a vibration wave monitor 27. The pre-embedded optical fibers 24 are provided in various directions in the tunnel sample 19 and between the linings 20 of the composite lining for measuring the catastrophe process of the tunnel sample 19 and the composite lining during the test. The water pressure sensors 25 are placed between the inner pressure-bearing sealing frame 16 and the physical model of the composite-lined fault tunnel for measuring the water pressure. The pressure sensors 26 are provided between the oil cylinder 15 and the pressure head 17 for monitoring the pressure and creep displacement. The vibration wave monitor 27 is pre-embedded in the tunnel sample 19 and provided between the linings 20 of the composite lining for measuring vibration waves.

The method of the first embodiment of the present application is as follows:

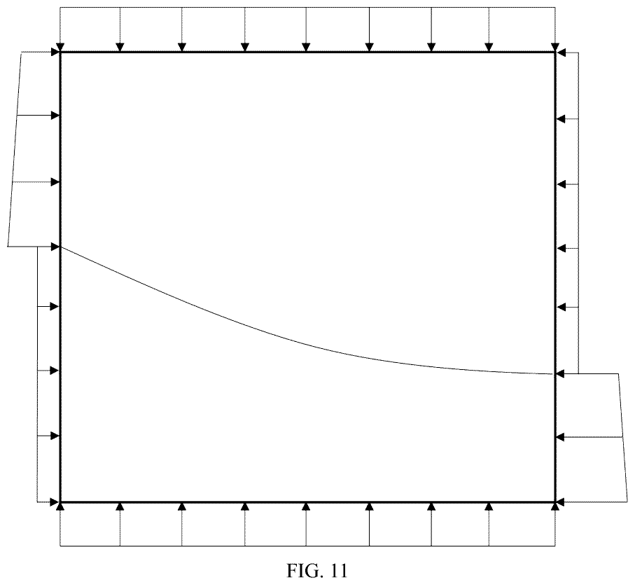

A method for simulating the activation of normal and reverse faults in water-rich tunnels induced by earthquakes and three-dimensional in-situ stress, using the above-mentioned centrifuge test device for complex fault activation in water-rich tunnels induced by earthquakes/creep, includes:
Step 1: Making a tunnel sample 19 with a horizontal fault strike;
Step 2: Installing the lining 20, the sheath 21, the sensor and the monitoring camera into the tunnel sample 19 to form a physical model of the composite-lined fault tunnel, injecting an appropriate amount of water into the space formed by the sheath, and then installing inner lining plugs on both sides of the sheath to ensure that the inner lining plugs completely seal the tunnel;
Step 3: Using lifting device to load the physical model of the composite-lined fault tunnel into the inner sealing frame, and selecting a suitable backing plate 18 so that a fault dip angle of the tunnel 14 sample meets the test requirements after the physical model of the composite-lined fault tunnel is loaded into the inner sealing frame;
Step 4: Injecting water into the inner pressure-bearing sealing frame 16, then covering the top plate of the inner pressure-bearing sealing frame 16 tightly, making the pressure head 17 in close contact with the physical model of the composite-lined fault tunnel, and checking the tightness of the inner pressure-bearing frame;
Step 5: Covering the top cover of the outer frame tightly, and ensuring that the upper oil cylinder is connected with the upper pressure head of the inner sealing frame;
Step 6: Starting the oil cylinder 15 on the true triaxial loading device for complex fault tunnels to apply pre-pressure to the physical model of the composite-lined fault tunnel;
Step 7: Reading data of the wireless sensor and the monitoring camera to determine whether the wireless sensor and the monitoring camera are working normally;
Step 8: Applying confining pressure, the three-dimensional stress is as shown in FIG. 11, where the lateral shear force increases from top to bottom along the normal direction, and the normal direction is uniform stress, accelerating the creep stage;
Step 9: After applying the confining pressure, starting the exciter 5 of the vibration table, the control terminal controlling the vibration frequency of the vibration table to be 90 Hz, making the vibration table surface vibrate at high frequency through the exciter, and the vibration table surface 8 applying earthquake waves to the physical model of the composite-lined fault tunnel to simulate the activation of normal and reverse faults in water-rich tunnels induced by earthquakes and three-dimensional in-situ stress;

Step 10: After the physical model of the composite-lined fault tunnel reaches the expected test condition or is damaged, ending the test, turning off the vibration table to reduce the pressure to zero, and overhauling the device for next use;

Step 11: Analyzing and processing according to the detection data recorded by the sensors and the monitoring camera.

The method of the second embodiment of the present application is as follows:

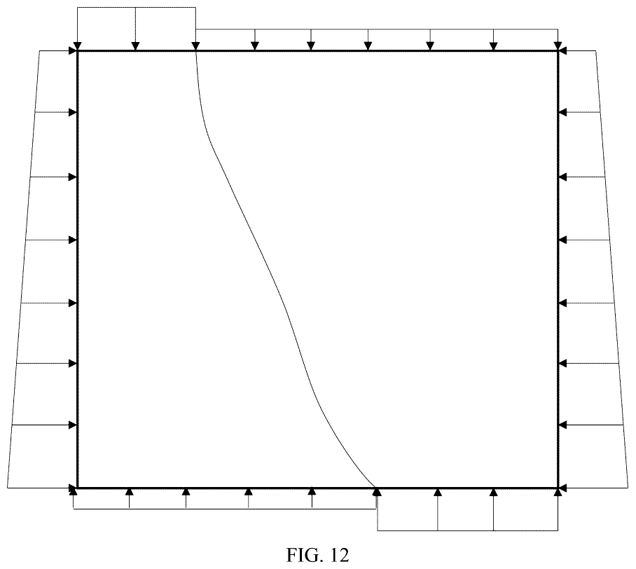

A method for simulating the activation of horizontal faults in water-rich tunnels induced by overweight and three-dimensional stress, using the above-mentioned centrifuge test device for complex fault activation in water-rich tunnels induced by earthquakes/creep, includes:
Step 1: Making a tunnel sample 19 with a normal fault;
Step 2: Installing the lining 20, the sheath 21, the sensor and the monitoring camera into the tunnel sample 19 to form a physical model of the composite-lined fault tunnel, injecting an appropriate amount of water into the space formed by the sheath, and then installing inner lining plugs on both sides of the sheath to ensure that the inner lining plugs 22 completely seal the tunnel;
Step 3: Using lifting device to load the physical model of the composite-lined fault tunnel into the inner sealing frame, and selecting a suitable backing plate 18 so that the fault dip angle of the tunnel 14 sample meets the test requirements after the physical model of the composite-lined fault tunnel is loaded into the inner sealing frame;
Step 4: Injecting water into the inner pressure-bearing sealing frame 16, then covering the top plate of the inner pressure-bearing sealing frame 16 tightly, making the pressure head 17 in close contact with the physical model of the composite-lined fault tunnel, and checking the tightness of the inner pressure-bearing frame;
Step 5: Covering the top cover of the outer frame tightly, and ensuring that the upper oil cylinder is connected with the upper pressure head of the inner sealing frame;
Step 6: Starting the oil cylinder 15 on the true triaxial loading device for complex fault tunnels to apply pre-pressure to the physical model of the composite-lined fault tunnel, the pre-pressure should be greater than that in step 7 to prevent the overall sliding of the physical model of the composite-lined fault tunnel during the centrifuge test;
Step 7: Applying confining pressure according to the test needs, as shown in FIG. 12, where the lateral stress increases from top to bottom along the normal direction, and the shear force is applied in the normal direction;
Step 8: After applying the confining pressure, starting the centrifuge main body 1, applying a rotation speed within 10 g, and observing whether there are potential safety hazards in the test;
Step 9: Reading data of the wireless sensor and the monitoring camera to determine whether the wireless sensor and the monitoring camera are working normally;
Step 10: Applying a rotation acceleration of 80 g to the centrifuge main body 1 according to the test plan to simulate the fault activation of the tunnel sample 19 and creep along the fault;
Step 11: Slowly increasing the rotation speed of the centrifuge main body 1 to the target speed to simulate the activation of horizontal faults in water-rich tunnels induced by overweight and three-dimensional stress;
Step 12: After the physical model of the composite-lined fault tunnel reaches the expected test condition or is damaged, ending the test to reduce the pressure to zero, turning off the centrifuge, and overhauling the device;
Step 13: Analyzing and processing according to the detection data recorded by the sensors and the monitoring camera.

Although the embodiments of the present application have been shown and described, those skilled in the art can understand that various changes, modifications, substitutions and variations can be made to these embodiments without departing from the principle and purpose of the present application, and the scope of the present application is limited by the claims and their equivalents.

What is claimed is:

1. A centrifuge test device for complex fault activation in water-rich tunnels induced by earthquakes/creep, comprising an overweight loading system, an earthquake wave loading system, a true triaxial loading device for complex fault tunnels, a physical model of a composite-lined fault tunnel, and a comprehensive monitoring system for deformation-stress-vibration;
the overweight loading system comprises a centrifuge main body, a test basket, and a counterweight block;
the earthquake wave loading system comprises a lower vibration table base, a lower exciter, a lower vibration table linear guide rail, a lower vibration table linear guide slider, an upper vibration table base, an upper exciter, an upper vibration table linear guide rail, an upper vibration table linear guide slider, a vibration table surface, and a stop block;
the true triaxial loading device for complex fault tunnels comprises a main frame, a top cover, an oil cylinder, an inner pressure-bearing sealing frame, a pressure head, and a backing plate;
the physical model of the composite-lined fault tunnel comprises a tunnel sample, a lining, a sheath, and an inner lining plug;
the comprehensive monitoring system for deformation-stress-vibration comprises a pre-embedded optical fiber, a water pressure sensor, a pressure sensor, and a vibration wave monitor;
the centrifuge test device for complex fault activation in water-rich tunnels induced by earthquakes/creep activates a fault of the tunnel sample by adjusting a pressure on the upper and lower sides of the tunnel sample, further causing a tunnel sample to creep along the fault;
the centrifuge main body provides overweight centrifugal force through high-speed rotation; the test basket is provided on both sides of the centrifuge main body;
the counterweight block and the vibration table are respectively provided on the two test baskets; the lower vibration table base is fixed on the test basket of the centrifuge;
the lower exciter is provided and fixed on the lower vibration table base, with both ends attached to the upper vibration table base; the lower vibration table linear guide rail is provided and fixed on the lower vibration table base; one end of the lower vibration table linear guide slider is provided on the lower vibration table linear guide rail, and another end of the lower vibration table linear guide slider is provided and fixed on a lower part of the upper vibration table base;
the upper vibration table base is connected to a table surface of the lower vibration table through the lower vibration table linear guide rail; the upper exciter is provided and fixed on the upper vibration table base, with both ends attached to a table surface of the upper vibration table;
the upper vibration table linear guide rail is provided and fixed on the upper vibration table base; one end of the upper vibration table linear guide slider is provided on the upper vibration table linear guide rail, and another end of the upper vibration table linear guide slider is provided and fixed on the lower part of the table surface of the upper vibration table;
the table surface of the upper vibration table is connected to the upper vibration table base through the upper vibration table linear guide slider; the table surface of the upper vibration table is connected to the true triaxial loading device for complex fault tunnels.

2. The centrifuge test device for complex fault activation in water-rich tunnels induced by earthquakes/creep according to claim 1, wherein the main frame is provided on the table surface of the vibration table; the top cover is provided above the main frame; the oil cylinder is provided and fixed in a reserved space of the main frame and the top cover;
multiple oil cylinders are provided on each side; the inner pressure-bearing sealing frame is provided in a space formed by the outer frame to form a closed sample placement space;
the pressure head is provided and fixed in a reserved hole of the inner pressure-bearing seal and connected to the oil cylinder; and
the backing plate is provided in the inner pressure-bearing sealing frame in close contact with the tunnel sample.

3. The centrifuge test device for complex fault activation in water-rich tunnels induced by earthquakes/creep according to claim 1, wherein the tunnel sample is a scaled-down model of a simulated tunnel containing faults provided in the inner pressure-bearing sealing frame;
the lining is provided in the tunnel sample in close contact with the tunnel sample;
the sheath is provided in the tunnel in close contact with the lining; the inner lining plug is provided in a space formed by the sheath to form a closed space with the sheath to isolate internal water from external water;
the wireless sensor is provided between the tunnel sample and the lining, and between the lining and the sheath; and
a monitoring camera is provided in the inner lining plug.

4. The centrifuge test device for complex fault activation in water-rich tunnels induced by earthquakes/creep according to claim 1, wherein the comprehensive monitoring system for deformation-stress-vibration monitors a deformation, a stress, and a response under vibration waves of the physical model of the composite-lined fault tunnel;
the pre-embedded optical fiber is provided in various directions in the tunnel sample and between layers of a composite lining;
the water pressure sensor is placed between the inner pressure-bearing sealing frame and the physical model of the composite-lined fault tunnel;
the pressure sensor is provided between the oil cylinder and the pressure head;
the vibration wave monitor is pre-embedded in the tunnel sample and provided between the layers of the composite lining.

5. An experimental method of the centrifuge test device for complex fault activation in water-rich tunnels induced by earthquakes/creep according to claim 1, comprising:
Step 1: making the tunnel sample;
Step 2: installing the lining, the sheath, the sensor, and the monitoring camera into the tunnel sample, injecting an appropriate amount of water into a space formed by the sheath, then installing inner lining plugs on both sides of the sheath to form the physical model of the composite-lined fault tunnel, ensuring that the inner lining plugs completely seal the tunnel;
Step 3: using lifting device to load the physical model of the composite-lined fault tunnel into the inner sealing frame, and selecting a suitable backing plate, so that a fault dip angle of the physical model of the composite-lined fault tunnel after being loaded into the inner sealing frame meets test requirements;
Step 4: injecting water into the inner pressure-bearing sealing frame, covering the top plate of the inner pressure-bearing sealing frame tightly, making the pressure head in close contact with the physical model of the composite-lined fault tunnel, and checking a tightness of the inner pressure-bearing frame;
Step 5: covering the top cover of the outer frame tightly, and ensuring that the upper oil cylinder is connected to an upper pressure head of the inner sealing frame;
Step 6: if simulating an activation phenomenon of normal and reverse faults in water-rich tunnels induced by earthquakes and three-dimensional in-situ stress, the following steps are adopted:
a. starting the oil cylinder on the true triaxial loading device to apply pre-pressure to the physical model of the composite-lined fault tunnel;
b. reading the data of the wireless sensor and the monitoring camera to determine whether the wireless sensor and the monitoring camera are working normally;
c. applying confining pressure, applying the same confining pressure on upper and lower sides through the oil cylinder on the upper and lower sides, and applying confining pressure that increases from top to bottom due to different depths in other directions;
d. after applying the confining pressure, observing a creep of the physical model of the composite-lined fault tunnel, starting the exciter of the vibration table after a period of time, making a table surface of the vibration table vibrate at high frequency through the exciter, and the table surface of the vibration table applying earthquake waves to the physical model of the composite-lined fault tunnel to simulate the activation of normal and reverse faults in water-rich tunnels induced by earthquakes and three-dimensional in-situ stress;
Step 7: if simulating an activation phenomenon of horizontal faults in water-rich tunnels induced by overweight and three-dimensional stress, the following steps are adopted:
a. starting the oil cylinder on the true triaxial loading device to apply pre-pressure to the physical model of the composite-lined fault tunnel to prevent an overall sliding of the physical model of the composite-lined fault tunnel during a centrifuge test;
b. applying confining pressure according to a test plan;
c. after applying the confining pressure, reading the data of the wireless sensor and the monitoring camera to determine whether the wireless sensor and the monitoring camera are working normally;
d. starting the centrifuge, applying a rotation speed within 10 g, and observing whether there are potential safety hazards in the test;
e. slowly increasing a rotation speed of the centrifuge to a target speed to simulate the activation of horizontal faults in water-rich tunnels induced by overweight and three-dimensional stress;
Step 8: after the physical model of the composite-lined fault tunnel reaches an expected test condition or is damaged, the experiment is ended, and stopping the earthquake table and the centrifuge, to reduce the pressure to zero, turning off the centrifuge and the vibration table, turning off the true triaxial loading device, and overhauling;
Step 9: analyzing and processing according to detection data recorded by the wireless sensor and the monitoring camera.
